(19) United States
(12) Patent Application Publication
(10) Pub. No.: US 2010/0318010 A1
(43) Pub. Date: Dec. 16, 2010

(54) ADJUSTABLE POSTERIOR SPINAL ORTHOSIS

(76) Inventors: Alan T. Sandifer, Casselberry, FL (US); Shannon R. Schwenn, Deltona, FL (US)

Correspondence Address: SNELL & WILMER LLP (OC), 600 ANTON BOULEVARD, SUITE 1400, COSTA MESA, CA, 92626, US

(21) Appl. No.: 12/738,276

(22) PCT Filed: Oct. 10, 2008
(86) PCT No.: PCT/US08/79592

§ 371 Date: Aug. 31, 2010

Related U.S. Application Data
(60) Provisional application No. 60/999,308, filed on Oct. 17, 2007.

(51) Int. Cl. A61F 5/00

(52) U.S. Cl. 602/19

(57) ABSTRACT

A spinal orthosis provides a lightweight posterior spinal member with a pair of flexible strap members removably attached at the top of the spinal member and secured and anchored at the bottom. Flexible straps form not only the shoulder straps but also contribute an adjustably fastening arrangement of smaller loops with connecting straps to abdominal pads. The abdominal pads can be fastened together and fastening pads can both secure and tighten the brace on the patient by pulling the flexible straps.

### RELATED APPLICATIONS

The present application claims priority of U.S. Provisional Patent Application No. 60/999,308 filed Oct. 17, 2007.

### BACKGROUND OF THE INVENTION

1. Field of the Invention

The present invention is directed to a lightweight spinal brace or othopraxis device for treatment of patients with upper back pain, strain, osteoporosis and compression fractures and more particularly, to an easily worn and adjustable extension compression posterior spinal orthosis for applying extension and compression forces in the treatment of spinal disorders.

2. Description of Related Art

The prior art discloses various devices to provide an orthotic treatment to relieve pain and to provide compensation for various types of spinal disorders such as multiple compression fractures that may occur from osteoporosis and kythotic postural changes.

Frequently, these spinal braces have employed a triangular or three point force vector system that includes an anchor point centrally located across the chest of the user which can be provided by a sternal pad supported, for example, by a metal frame and a corresponding anchor point on the lower portion of the abdomen, provided by a pubic pad supported by a frame. Thus, two forward pressure points are provided on the front of the user and a centrally located adjustable lumbar pad is provided on the user's back, again adjustably fixed to a metal frame so that three force vectors can be applied to create a condition of hyperextension on the spinal column, while permitting some mobility to the user. An example can be seen in the Jewett brace shown in U.S. Pat. No. 3,274,996.

Basically, this orthotic modality of treatment involves an anterior spinal hyperextension orthosis to create spaced anterior anchor stabilization points across the sternum and pubic areas with an adjustable force applied at an intermediate position on the user's back. Numerous variations of this treatment can be found, for example in U.S. Pat. No. 5,599,287, U.S. Pat. No. 6,190,343, U.S. Pat. No. 6,790,191, U.S. Pat. No. 6,010,472, U.S. Pat. No. 5,674,187, and U.S. Pat. No. 3,945,376. Other examples and variations can also be found in the orthodesic art.

An article in the American Journal of Physical Medicine and Rehabilitation “Effects of a New Spinal Orthosis of Posture, Trunk Strength And Quality of Life In Women With Postmenopausal Osteoporosis,” Volume 83, No. 3, Pages 177-186, March 2004, described a study on the management of vertebra fractures caused by osteoporosis. A thoracolumbar orthosis disclosed in the study included a narrow back pad centered along the spine which could be workable as cold material to adjust to the particular patient, a system of belts, and an abdominal pad to apply lower, intermediate and upper forces on the back pad to mold it to the spinal region of the user. See U.S. Pat. No. 6,063,047, U.S. Pat. No. 2,828,737, U.S. Pat. No. 1,755,641 and U.S. Pat. No. 1,581,791.

There is still a need in the orthopedic field to provide an economical lightweight, comfortably wearing extension compression orthosis and method to treat mid spinal pathologies that does not require cumbersome strapping arrangements. Such a brace should be easily adjusted by the user to accommodate for various daily activity demands, to provide an improved, firm, lightweight posterior support structure that is easy to apply and that permits vector forces to be applied to the body by the user and to permit adjustments to match the size and configuration of the user.

### SUMMARY OF THE INVENTION

The present invention provides a lightweight spinal orthosis that can be adjustably mounted, for example by an orthotist in an economical manner so that it can be easily placed on a user's body to apply extension and compression forces in the treatment of spinal disorders. As can be appreciated, various medical issues and spinal problems can occur frequently with aging. The present orthosis can be economically manufactured and easily placed on and removed from the user or patient, preferably similar to putting on a clothing jacket. A rigid posterior frame or spinal member can be formed from a stamped metal plate or from a molded plastic and can be further subjectively configured to meet the demands and requirements of the specific patient.

A pair of flexible strap members, attached adjacent an upper portion of the posterior spinal member, and attached adjacent a lower portion of the spinal member are provided on opposite lateral sides of the spinal member, and have a sufficient length to provide adjustable shoulder straps for the user. Thus, the shoulder straps can be conveniently adjusted to permit the user's arms to extend through the shoulder straps and then abdomen or waist closure pads are fastened together across the abdominal area of the patient and subsequently tighten to draw the flexible strap members against the patient's body.

The waist closure pads can comprise a pair of flexible abdomen support members, having for example, a connecting strap attached to respective smaller loops of the flexible strap members located below the loops for accommodating shoulder straps. The flexible abdomen support members can adhere together and connecting straps with pocketed finger tabs can be adjustably fastened across the surface of the flexible abdomen support members and each other to secure the spinal member on the user while pulling the flexible strap members.

The pair of flexible strap members can be removably attached adjacent an upper portion of the spinal member and permanently attached to a lower portion of the spinal member and adjustments can be easily made by an orthotist to both the flexible strap members and the connecting straps in order to size the spinal orthosis to a user.

The posterior spinal member is approximately of a length to extend across the user's thoracic to the coccyx of the user's spine. The posterior spinal member can have an elongated triangular configuration with apertures to reduce weight. The spinal member can be formed from a thin metal plate and bent to increase strength while configured to the user's spine. Alternatively, the spinal member can be molded from a plastic resin.

A replaceable resilient pad of a nap material on one side and an apertured texture to permit air flow can be removably attached to an internal surface of the posterior spine member for contacting the user's back.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings.

FIG. 1 is a front perspective view of the posterior spinal orthosis of the present invention on a user;

FIG. 2 is an elevated rear view of the spinal orthosis;

FIG. 3 is a rear view of the present invention on a user;

FIG. 4 is a side view of the present invention on a user;

FIG. 5 is a partial side view of the side strapping configuration;

FIG. 6 is a perspective view of a user beginning the donning of the orthosis by closing the abdomen support members;

FIG. 7 shows the user tightening the spinal orthosis by placing his fingers in pocketed finger pull tabs at the end of tightening straps;

FIG. 8 is a perspective view disclosing an adjustment of a tightening strap fitting on a user; and

FIG. 9 is a perspective view disclosing an adjustment of flexible strap members for adjustment to a user.

### DETAILED DESCRIPTION OF THE PREFERRED EMBODIMENTS

Reference will now be made in detail to the preferred embodiments of the invention which set forth the best modes contemplated to carry out the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well known methods, procedures and components, and have not been described in detail as not to unnecessarily obscure aspects of the present invention.

In the preferred embodiment of the present invention as described herein, it should be appreciated that various modifications could be made by a person of ordinary skill in this field. It is contemplated that the spinal frame of the present invention can be made from a thin metal plate and an orthotist can subsequently adjust the curvature of the plate to accommodate a particular patient's spine. Alternatively, the spinal frame could be a molded plastic member. Additionally, the strapping system is also capable of adjustment to match a particular body shape of the patient.

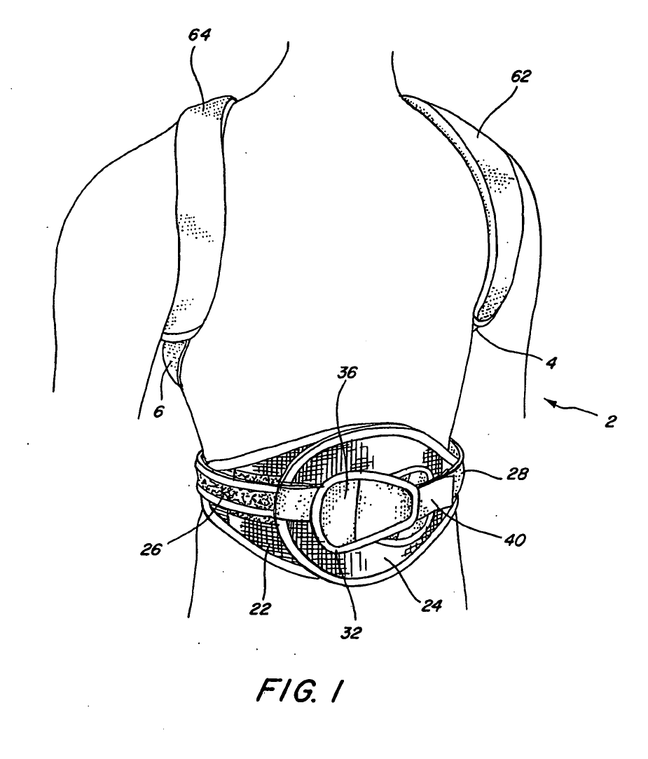

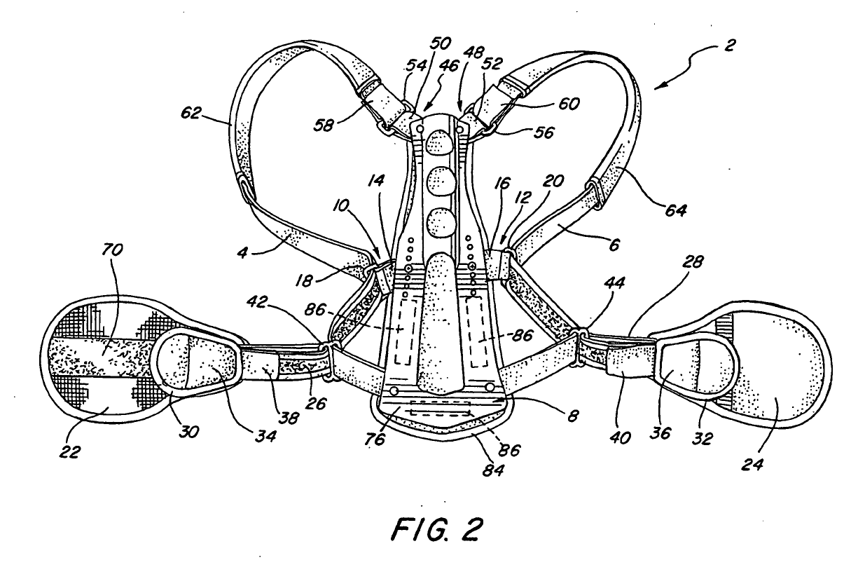

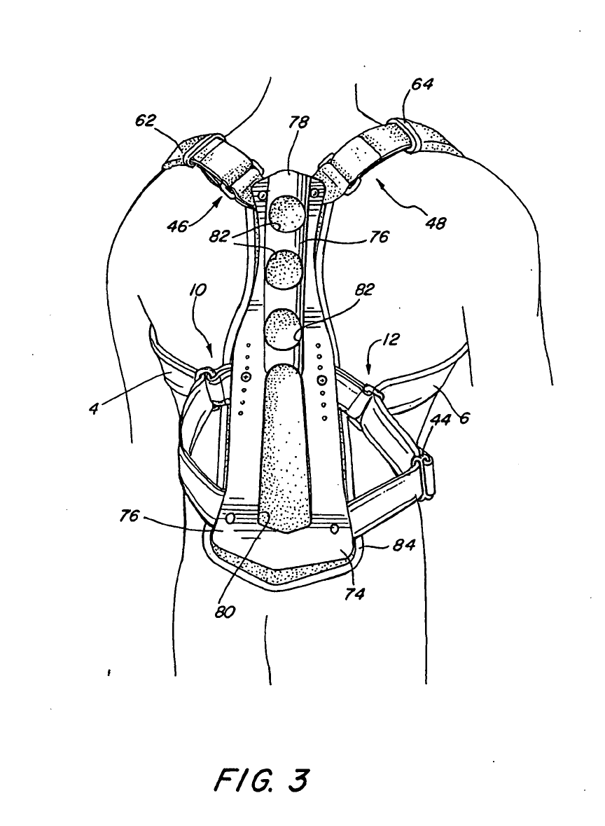

Referring to FIGS. 1 and 2, a spinal orthosis 2 of the present invention has a posterior spinal member 8 with a frame member 74 either of a bent and curved metal plate or a molded plastic resin part.

A flexible resilient pad 84 is adhered on an internal surface of the spinal frame member 74 to provide a comfortable cushioned fit for the user. Elongated flexible strap members 4 and 6 are removably attached at a top portion of an upper edge of a curved neck 78 of the spinal frame member 74. Upper attachment members 46 and 48 of appropriate looped straps 50 and 52 are fastened by a screw or rivet as seen in FIG. 2 adjacent the top of the curved neck 78. The respective straps 50 and 52 anchor an upper attachment loop 54 and 56, respectively that can also support spinal adjustment straps 58 and 60.

The elongated flexible strap members 4 and 6 can be removably fastened to the respective loops 54 and 56. The strap members 4 and 6 extend along the lateral sides of the spinal frame member 74. The curved neck 78 extends down to an intermediate portion of the spinal frame member 74 and intermediate attachment members 10 and 12 are mounted on either side, in a width direction, of the spinal frame member 74. Looping attachment straps 14 and 16 are appropriately fastened, for example, by rivets to the frame member 74 and capture attachment loops 18 and 20, respectively.

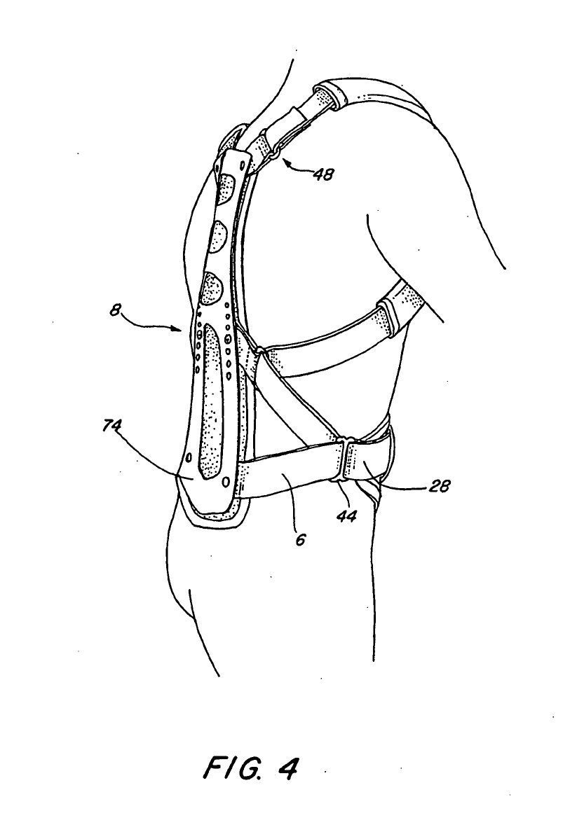

The flexible strap members 4 and 6 are journaled through the respective attachment loops 18 and 20 and are permanently fixed at a spinal triangular base 76 of the spinal frame member 74, for example by a rivet or other fastener, see FIGS. 2 and 4.

Each of the flexible strap members 4 and 6 are configured to provide upper major loops that function as shoulder straps for a user or patient. Tubular shoulder pads 62 and 64 are mounted on the respective strap members 4 and 6 within the upper major loops to cushion any force on the patient's shoulders. Minor lower loops of the strap members 4 and 6 are further created between the riveted ends of the respective flexible strap members 4 and 6 and the attachment loops 18 and 20, respectively to enable securing the posterior spinal member triangular base 78 against the user's lower back as shown in FIG. 4.

Flexible padded abdominal support members 22 and 24 support connecting straps 26 and 28, respectively, that extend through respective double loops 42 and 44 on either side of the posterior spinal member 8. Thus, the abdominal support members 22 and 24 can slide with the connecting straps 26 and 28 along the minor loops formed by the flexible strap members 4 and 6, see FIGS. 2, 4 and 5.

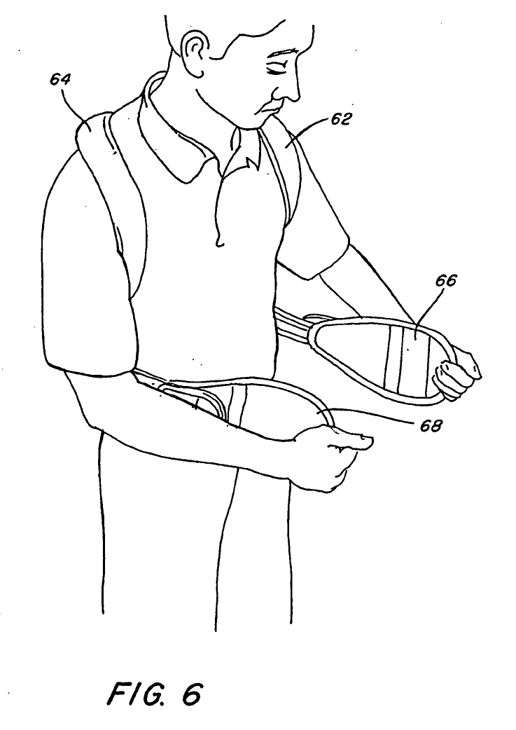

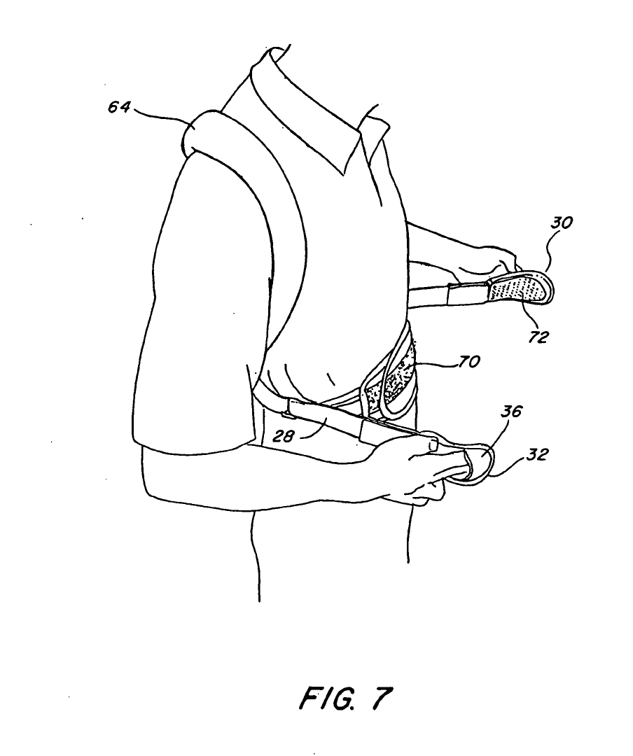

As can be seen in FIG. 6, the respective abdomen support members 22 and 24 can be pulled and loosely fastened across an abdomen section of the user, as shown in FIG. 7. An abdomen support swatch of hook material 66, for example of Velcro® can be located on an inner surface of each abdomen support member 22 and 24. Correspondingly, the outside surface of each support member 22 and 24 can have a nap fabric covering 68. As a result, a subjective fastening, in an overlapping manner, of the abdomen support members 22 and 24 can be performed by the user after the user's arms are inserted through the shoulder straps.

Additionally, as seen in FIG. 7, fastening pads 30 and 32 with exterior pockets 34 and 36 respectively, of a size to receive one or more fingers, are attached at the other end of the respective connecting straps 26 and 28. Due to the size of the abdomen support members 22 and 24 and the fastening pads 30 and 32, the straps 26 and 28 are captured in one of the loops of the respective double loop members 42 and 44. The underside of each of the fastening pads 30 and 32 have a fastening hook material 72 capable of engaging the napped or looped fabric of the reinforced straps 26 and 28 and an abdomen swatch of nap material 70 on an exterior surface of the abdomen support member 22.

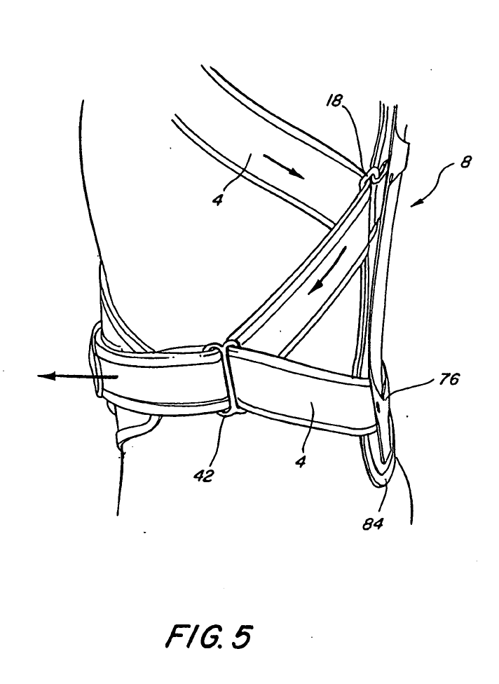

Thus, as seen in FIG. 7, pulling the respective fastening pads 30 and 32 tightens the waist connecting straps 26 and 28 and pulls the smaller loops of the flexible strap members 4 and 6, and as seen in FIG. 5, will also tighten the shoulder straps of flexible strap members 4 and 6 to pull the user's shoulders backwards for a snug fit against the posterior spinal member 8. The fastening pads 30 and 32 can adhere to each other and also the nap material of the abdomen support members 22 and 24.

As can be appreciated, the length of the respective strap members 4 and 6 can be appropriately adjusted by an orthotist, as seen in FIG. 9, by cutting the ends of the straps which have an exterior loop fabric texture and then capturing the cut end within the respective upper adjustment straps 58 and 60. Thus, as is shown in FIG. 9, the free end of the flexible strap member 4 is cut to size for a particular patient and then secured in a sandwich like manner by the hook material provided on and between the upper adjustment straps 58.

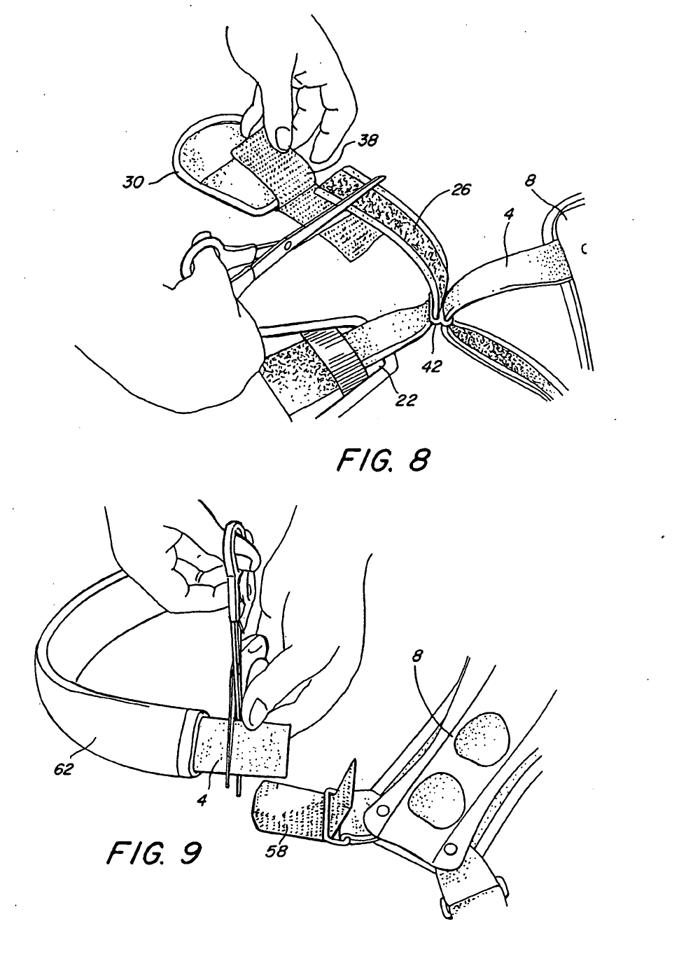

As can be seen in FIG. 8, a similar size adjustment procedure can be performed by an orthotist in cutting, for example, the connecting strap 26 at an end adjacent the fastening pad 30. The abdomen adjustment straps 38 and 40 also have a hook material on their interior surfaces and adhere to the napped fabric on the exterior of the connecting straps 26 and 28, respectively, when secured in a sandwich manner over the ends of the adjustment straps. Thus, the present invention permits two areas of adjustment by cutting or removing unwanted lengths of the respective fastening strap members 4 and 6 and the respective connecting straps 26 and 28.

The spinal frame member 74 has a cross sectional curved neck portion 78 to provide rigidity and strength to the reduced width at the top of the spinal frame member 74 while also curved along a longitudinal axis to accommodate the curve of the spine. A series of holes 82 can be provided for reducing weight. The lower triangular base 76 also has an elongated central slot 80, also to reduce weight. The lower tip of base 76 can be flared outward at the base of the spine. The resilient spinal pad 84 has a napped fabric on one side specifically designed for adhering to a hook material, for example of a Velcro® brand. Swatches of Velcro® hook material 86 can be appropriately adhered along an interior surface of the spinal frame 74 as shown in dotted lines in FIG. 2, as an illustration. The other side of the resilient spinal pad 84 can have a pocked or apertured textured surface configuration to assist in aerating the length of the resilient spinal pad 84 that will bear against a user. The spinal pad 84 is removable from the hook swatches 86 and washable for hygienic reasons.

After an orthotist has appropriately sized the length of the pair of flexible strap members 4 and 6 and likewise the length of the connecting straps 26 and 28 as shown in FIGS. 8 and 9, the spinal orthosis 2 can then be easily donned by a user with limited mobility. The upper major loops formed by the flexible strap members 4 and 6 will be encompassed with the shoulder tube pads 62 and 64. The user can place their arms through these major loops so that the shoulder tube pads 62 and 64 are appropriately placed across the user's shoulders, as shown for example, in FIG. 6. The user can then close the respective abdomen support members 22 and 24 so that the loop and hook material can adhere the abdomen support members 22 and 24 together while the straps 4 and 6 are still in a relatively loose condition.

As shown in FIG. 7, the user can either grasp the fastening pads 30 and 32 and tighten the spinal orthosis against the spine as shown, for example, in FIG. 5, or can insert her/his fingers into the respective pockets 34 and 36 on the respective fastening pads 30 and 32 and by extending her/his arms, tighten the brace 2 to the spine of the user while pulling the flexible strap members 4 and 6. By pressing the hook surface of the fastening pads 30 and 32 against the nap material of the abdomen support member or the exterior nap surface of a corresponding fastening pad, the tightened position is then retained for wearing purposes.

In summary, the spinal orthosis of the present invention can position a posterior spinal member 8 so that its length extends across the thoracic to the adjacent coccyx of the user's spine. The arrangement of the fastening straps 4 and 6 provide not only an adjustable set of shoulder straps that can be easily donned by a user, but further contribute to the easy fastening of the flexible abdominal support members 24 and 22 together wherein extensions of the fastening pads 30 and 32 can removably secure the brace in position on the user's spine while specifically tightening the shoulder straps to draw the user's shoulders back against the posterior spinal member 8.

Those skilled in the art will appreciate that various adaptations and modifications of the just-described preferred embodiment can be configured without departing from the scope and spirit of the invention. Therefore, it is to be understood that, within the scope of the amended claims, the invention may be practiced other than as specifically described herein.

What is claimed is:

1. A spinal orthosis comprising:
a posterior spinal member for supporting a user's spine;
a pair of flexible strap members, attached adjacent an upper portion of the spinal member and attached adjacent a lower portion of the spinal member, the respective strap members can extend on opposite sides of the spinal member and have a sufficient length to provide shoulder straps for the user;
a pair of attachment members, on the spinal member, enabling a sliding movement for each flexible strap member, each attachment member is located along opposite sides of the spinal member for enabling the flexible strap members formation of shoulder straps adjacent the upper portion of the spinal member and providing smaller loops adjacent the lower portion of the spinal member; and
a pair of flexible abdomen support members, each having a connecting strap attached to a respective smaller loop of the flexible strap member wherein the flexible abdomen support members can adhere together and the connecting straps can be adjustably fastened to the flexible abdomen support member to secure the spinal member on the user.

2. The spinal orthosis of claim 1 wherein the flexible abdomen support members adhere together when overlapped.

3. The spinal orthosis of claim 2 wherein the flexible abdomen support members have one of a nap material and a hook material on a surface for adhering together.

4. The spinal orthosis of claim 1 wherein each connecting strap has a fastening pad at one end and an abdomen support member at the other end.

5. The spinal orthosis of claim 4 wherein the fastening pad secures to a surface of one of the flexible abdomen support members.

6. The spinal orthosis of claim 5 wherein the flexible abdomen support member has an outer surface with a complementary surface for securement to a fastening pad.

7. The spinal orthosis of claim 4 wherein the fastening pad includes a pocket to permit a user's finger and/or fingers to be captured for tightening the connecting strap.

8. The spinal orthosis of claim 7 wherein the fastening pad includes a pair of flexible straps that removably adhere to one end of the connecting strap.

9. The spinal orthosis of claim 1 wherein a loop fastener connects the connecting strap for sliding movement on the smaller loop of the flexible strap member.

10. The spinal orthosis of claim 1 wherein the posterior spinal member is a curved flat metal plate having a triangular base and a narrower curved upper portion.

11. The spinal orthosis of claim 10 wherein the posterior spinal member includes strips of hook material and a resilient pad is removably connected to the hook material.

12. The spinal orthosis of claim 11 wherein one surface of the resilient pad has a nap material and the opposite surface has an apertured texture to permit air flow.

13. The spinal orthosis of claim 1 wherein the pair of flexible strap members are attached to respectively cantilevered loops at the top of the spinal member and the flexible strap members have a removable strap that secures to both sides of each end of the strap members to enable adjustment and fit of a length of the strap members to the size of the user with the removable strap captured in the cantilevered loop.

14. A spinal orthosis comprising:
a posterior spinal member of a length to extend across the thoracic to adjacent the coccyx of the user's spine;
a pair of flexible strap members, attached adjacent an upper portion of the spinal member and attached adjacent a lower portion of the spinal member, the respective strap members can extend on opposite sides of the spinal member and have a sufficient length to provide shoulder straps for the user;
a pair of attachment loop straps, on the spinal member, enabling a sliding movement for each flexible strap member, each attachment loop strap is located along opposite sides of the spinal member for enabling the flexible strap members formation of shoulder straps adjacent the upper portion of the spinal member and providing smaller loops adjacent the lower portion of the spinal member; and
a pair of flexible abdomen support members of a size to collectively cover the user's abdomen, each having a connecting strap attached to a respective smaller loop of the flexible strap member wherein the flexible abdomen support members can adhere together and the connecting straps can be adjustably fastened to the flexible abdomen support member to secure the spinal member on the user.

15. The spinal orthosis of claim 14 wherein each connecting strap has a fastening pad at one end and an abdomen support member at the other end.

16. The spinal orthosis of claim 15 wherein the fastening pad secures to a surface of one of the flexible abdomen support members.

17. A spinal orthosis comprising:
a posterior spinal member of a length to extend across the thoracic to adjacent the coccyx of the user's spine;
a pair of flexible strap members, removably attached adjacent an upper portion of the spinal member and permanently attached adjacent a lower portion of the spinal member, the respective strap members can extend on opposite sides of the spinal member and have a sufficient length to provide shoulder straps for the user;
a pair of attachment loop straps, on the spinal member, enabling a sliding movement for each flexible strap member, each attachment loop strap is located along opposite sides of the spinal member for enabling the flexible strap members formation of shoulder straps adjacent the upper portion of the spinal member and providing smaller loops adjacent the lower portion of the spinal member, each attachment loop strap has a strap portion anchored to a side of the posterior spinal member with a loop member flexibly supported at the end of the strap portion; and
a pair of flexible abdomen support members of a size to collectively cover the user's abdomen, each having a connecting strap attached to a respective smaller loop of the flexible strap member wherein the flexible abdomen support members can adhere together and the connecting straps can be adjustably fastened to the flexible abdomen support member to secure the spinal member on the user.

18. The spinal orthosis of claim 17 wherein the pair of flexible strap members are attached to respectively cantilevered loops at the top of the spinal member and the flexible strap members have a removable strap that secures to both sides of each end of the strap member removably attached to the spinal member to enable adjustment and fit of a length of the flexible strap members to the size of the user with the removable strap captured in the cantilevered loop.

19. The spinal orthosis of claim 18 wherein each connecting strap has a fastening pad at one end and an abdomen support member at the other end.

20. The spinal orthosis of claim 19 wherein the fastening pad includes a pocket to permit a user's finger and/or fingers to be captured for tightening the connecting strap and the flexible abdomen support member has an outer surface with a complementary surface for securement to a fastening pad.
